(19) United States
(12) Patent Application Publication
(10) Pub. No.: US 2005/0020977 A1
(43) Pub. Date: Jan. 27, 2005

(54) Intravascular infusion site anti-tamper guard having means for site inspection

(75) Inventors: Rosa Lee Eldridge, Rose Hill, VA (US); David Jones, Knoxville, TN (US)

Correspondence Address: PITTS AND BRITTIAN P C, P O BOX 51295, KNOXVILLE, TN, 37950-1295, US

(73) Assignee: Alba Innovations, Inc., Knoxville, TN (US)

(21) Appl. No.: 10/627,888

(22) Filed: Jul. 25, 2003

(51) Int. Cl. A61M 5/00

(52) U.S. Cl. 604/111; 604/180

(57) ABSTRACT

A tamper resistant guard for protectively covering a patient's infusion site while allowing visual inspection of the site without manipulation of the guard. A resilient base panel includes an open portion therein that is positioned over the infusion site, allowing visual inspection of the dermal tissue proximal of the site for indications of infiltration and extravasation of medications and degeneration of dermal tissues proximal to the infusion site. A resilient flap having a flexible window therein is hingedly secured to the base panel with the window positioned in register with the panel open portion, to facilitate visual inspection of the infusion site without flap manipulation. The flexible window is positioned in covering relationship over the infusion site and includes means for fastening a flap attaching side in overlapping orientation to secure infusion tubing between the flap and base panel, thereby preventing patient tampering with the tubing and the infusion site.

### CROSS REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of Invention

The invention pertains to devices for stabilizing a catheter inserted at intravascular and intravenous infusion sites. More particularly, this invention pertains to a tamper resistant device for covering and securing a catheter inserted at an infusion site while providing rapid visual inspection of the infusion site and adjacent tissue for indications of infiltration and extravasation of medications.

2. Description of the Related Art

In the field of medical care, an urgent and rapidly growing need exists for protection of readily accessible intravenous infusion sites (i.e. dermal sites for injection of medication into veins), and/or intravascular infusion sites (i.e. dermal sites for injection of medication into arteries or veins), for young patients suffering from AIDS/HIV, cancer, or similar life-threatening diseases, and patients of about forty-five and older suffering from AIDS/HIV, cancer, or advanced delirium, dementia and/or Alzheimer's disease. These young and elderly patients typically require every readily accessible infusion site to be utilized for long-term infusion of AIDS/HIV treatment medications or cancer treatment medications. The medications utilized for treatment of AIDS/HIV, cancer, delirium, advanced dementia and/or Alzheimer's disease are typically highly caustic to the vascular walls and serve as a vesicant agent when allowed to infiltrate external of veins or arteries and into adjacent dermal and subdermal tissues. In addition, for young patients and elderly patients requiring treatment of chronic diseases, the frequently utilized veins tend to be reused and frequently deteriorate from use, which require medical practitioners to seek access to a multitude of additional infusion sites on each patient. After the preferred intravenous sites are used to the point of deterioration, then access to an infusion site into an artery is selected, with the requirement for hospitalization. Patients that receive continuous or intermittent infusion of caustic and/or vasoconstrictive medications for treatment of the above identified diseases can readily suffer from tissue degeneration and atrophy, ischemic necrosis and sloughing of tissue if the medications infiltrate from veins or arteries and into dermal tissue proximal of the infusion site(s). Infiltration of medications from a vein or artery due to a dislodged catheter can rapidly initiate: erythema venenation, edema, pain, and necrosis of the dermal and subdermal tissues proximal of the infusion site. Extensive tissue damage due to infiltration of medications into dermal and subdermal tissues is generally referred to as extravasation, and is typically caused by a catheter that is displaced from proper insertion in the target vasculature at the infusion site. For very young patients, the catheter may be displaced by unintended actions of the patient. For older patients that suffer from delirium, dementia, or are chemically sedated, and/or are partially restrained during treatment, there is a significant risk of the catheter being displaced from the infusion site by the unintended or confused actions of the patient. If the patient does not understand the benefits of maintaining a properly positioned catheter at an infusion site, the patient may disrupt the catheter and/or associated tubing at the infusion site. Infiltration of the medication(s) initiate tissue damage leading to extravasation of the patient's dermal and subdermal tissue. Therefore, the medical practitioner must frequently visually inspect the infusion site(s) for each patient under his/her care, with a minimum of disruption of the patient and preferably no movement of the infusion site cover. In addition, if visual inspection suggests infiltration leading to extravasation proximal of the infusion site, the medical practitioner must be provided rapid access to. the catheter inserted at the infusion site. Paradoxically, the catheter and tubing associated with the infusion site must be protected from unintentional or intentional tampering by the patient.

The preferred infusion site is selected to provide rapid access by medical practitioners to one or more of the patient's veins or certain arteries in emergency situations. The vein or artery selected typically provides rapid access for the medical practitioner for changing of tubes, medication storage bags and for inspecting the infusion site(s). The infusion site is also generally accessible to tampering by the patient. Typical infusion sites are located on the patient on the hand, wrist, forearm, elbow, upper arm, upper chest, groin, foot and ankle (typically utilized for the very young). If the patient is not aware of the benefits of keeping the infusion site intact, or is suffering from delirium, dementia, the patient may attempt to dislodge the catheter without the medical practitioner's knowledge.

If the infusion site is disrupted and the catheter is partially dislodged or withdrawn from the interior of the vein but remains within the subcutaneous tissue for as little as one to two hours, leakage identified as extravasation occurs. The medicinal fluids utilized for treatment of HIV/AIDS or cancer typically include vasoconstricting agents and/or caustic agents, with the extravasation of the medicinal fluids rapidly degrading surrounding perivascular tissue, subcutaneous tissue, and dermal tissue over approximately six hours to about twenty-four hours. Extravasation occurs due to the vasoconstrictive or caustic agent properties of a multitude of medications utilized for treatment of patients suffering from delirium, dementia, cancer, and/or AIDS or other chronic diseases. If extravasation of vasoconstrictive or caustic agents is not detected by frequent visual inspections of the infusion site, and treatment is not immediately provided for the dermal tissue undergoing degradation from exposure to the caustic agents (i.e. within approximately six hours to about twenty-four hours of initial infiltration), necrosis of the dermal and subdermal tissues can readily occur in twenty-four to forty-eight hours, leading to the onset of gangrene of tissue in about seven to ten days. Patients suffering from delirium, dementia or who are heavily sedated, will not readily identify detrimental changes to dermal tissues proximal of each infusion site. In order to protect the patient, rapid detection is needed by a medical practitioner conducting frequent visual inspections of each infusion site. Rapid and frequent visual inspections are facilitated only if a covering over each infusion site provides a clear view of each site without requiring physical adjustments for viewing of each site. If extravasation occurs, the infusion site at the location of infiltration is lost, and the potential infusion sites on the extremity proximal of the disrupted infusion site are typically not functional also.

To guard against extravasation of medication in very young and aged patients receiving medications including vasoconstrictive and/or caustic agents, most hospital protocols require visual inspections of each of one or more infusion site(s) for each patient by a medical practitioner on a recurring hourly basis or a similar schedule. The frequency of visual inspections can be overwhelming for a typical patient to medical staff ratio in many non-critical care facilities of about eight to ten patients to one medical practitioner. Further, the frequency of visual inspections are a real burden to the nursing staff even for a typical patient to medical staff ratio in many intensive care units of about two to three patients to one medical practitioner, if the hospital is adequately staffed.

It is preferred that a protective guard device is configured to be flexible and sized to encircle a portion of a patient's body for covering an infusion site to limit tampering by the patient. It is also preferred that a tamper resistant guard is provided with means for rapid visual inspection of the infusion site and the adjacent dermal surfaces without medical personnel having to manipulate the guard to uncover and recover the infusion site after visual inspection.

Therefore, a need exists for a tamper resistant device that covers and maintains the proper positioning of a catheter and associated tubing at an infusion site, while providing means for rapid visual inspection of the infusion site and surrounding dermal tissue by a medically trained professional.

### BRIEF SUMMARY OF THE INVENTION

According to one embodiment of the present invention, a tamper resistant guard is provided for covering an intravascular infusion site having a catheter inserted through a dermal surface of the patient. The tamper resistant guard provides for rapid visual inspection of the infusion site for indications of infiltration of fluids and disruption or degradation of dermal tissue proximal to the infusion site. The tamper resistant guard includes a base panel of sufficient size and flexibility to cover the infusion side and adjacent dermal tissue. An open portion is disposed in the base panel and is sufficiently sized to be positioned over the infusion site. The base panel further includes a receiving end having first and second means for attaching disposed respectively on an outer receiving surface and a dermal receiving surface. The open portion provides for visual inspection of the infusion site and adjacent dermal tissue without requiring adjustment of the base panel by a medical practitioner.

The panel open portion is positioned in covering relationship over the infusion site and is maintained against the patient's dermal surface proximal of the infusion site by means for retaining extended from the panel and configured to be releasably attached to the panel second means for attaching. A resilient flap having a flexible window therein is hingedly secured on the panel outer receiving surface. The flap flexible window is sized for positioning in register with the panel open portion. The flap includes an attaching side having means for fastening thereon for releasably securing the flap attaching side to the panel first means for attaching on the outer receiving surface with the window in register with the panel open portion and positioned in covering relationship over the infusion site. The flap attaching side and means for fastening is releasably secured to the panel first means for attaching in an overlapping relationship with the panel to cover and retain therebetween a selected length of tubing extended from the infusion site. The tamper resistant guard provides a covering relationship over the infusion site and provides an overlapping relationship with the panel and a selected length of tubing extended from the infusion site to minimize patient disruption of the infusion site and associated tubing. The tamper resistant guard allows rapid visual inspection of the infusion site through the flap flexible window and panel open portion by the medical practitioner to provide early detection for infiltration and extravasation of medicinal fluids and treatment for degeneration of dermal tissue proximal to the infusion site.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 is an exploded view of a tamper resistant guard of the current invention, illustrating a flexible base panel having an opening therein and a flexible flap having a resilient window therein;

FIG. 2 is a perspective view of the flexible flap attached to the outer receiving surface of the base panel of FIG. 1, with the base panel positioned in an overlapping configuration and having a portion of tubing secured between the flap and the base panel;

FIG. 3 is an exploded view of an alternative embodiment of FIG. 1, illustrating a flexible flap having a flexible window therein and a second flap positioned to be attached to by surfaces with hook and loop fastening means thereon;

FIG. 4 is a perspective view of the flexible flap and second flap attached to the outer receiving surface of the base panel of FIG. 3, with the base panel positioned in an overlapping configuration and having a portion of tubing secured between the flap and the base panel;

FIG. 5a is a perspective view of a tubing tie having a fastening material thereon for releasably securing the tie to the base panel;

FIG. 5b is a perspective view of a tubing tie positioned to encircle a portion of tubing to be secured between the flap and the base panel;

FIG. 6 is a cross-section view of the guard base panel in an overlapping configuration, with a portion of tubing positioned between the base panel and the overlying flap;

FIG. 7 is a side view of a medial portion of a tamper resistant guard having a resilient flap and window protectively covering a forearm infusion site;

FIG. 8 is a side view of a distal portion of the guard of FIG. 7, illustrating a flap attaching side covering tubing ties and tubing extended from the forearm infusion site;

FIG. 9 is a side view of a tamper resistant guard having a resilient flap and window protectively covering an antecubital infusion site;

FIG. 10 is a side view of a distal portion of the guard of FIG. 9 having a flap attaching side covering a tubing ties and tubing extended from the antecubital infusion site;

FIG. 11 is a side view of an alternative embodiment of the tamper resistant guard of FIG. 9, illustrating a resilient flap and window covering an antecubital infusion site and two retaining straps encircling the elbow;

FIG. 12 is a side view of a distal portion of the guard of FIG. 11 having a flap window covering a second antecubital infusion site and a flap attaching side covering two tubes extended from two infusion sites;

FIG. 13 is a side view of a medial portion of a tamper resistant guard having a resilient flap and window covering an upper arm infusion site;

FIG. 14 is a side view of a distal portion of the guard of FIG. 13, illustrating a flap attaching side covering tubing connected to the upper arm infusion site;

FIG. 15 is a side view of a tamper resistant guard having a resilient flap and window covering an outer upper arm infusion site;

FIG. 16 is a side view of a medial portion of the guard of FIG. 15 having a flap attaching side covering tubing extended from the upper arm infusion site and with two retaining straps encircling the elbow;

FIG. 17 is a perspective view of a tamper resistant glove guard having a resilient flap and window covering an infusion site on the back side of a hand;

FIG. 18 is a perspective view of the palmar portion of the glove guard of FIG. 17 illustrating a flap attaching side extendable to cover a plurality of tubing ties and tubing extended from the hand infusion site;

FIG. 19 is a perspective view of a tamper resistant glove guard having a resilient flap and window covering a carpal infusion site;

FIG. 20 is a perspective view of the palmar portion of the glove guard of FIG. 19, illustrating a flap attaching side covering a plurality of tubing ties and tubing extended from the carpal infusion site;

FIG. 21 is a perspective view of a tamper resistant guard having a resilient flap and window covering a subclavian infusion site;

FIG. 22 is a perspective view of the back of a patient illustrating the means for retaining for maintaining the guard in covering relationship over the subclavian infusion site;

FIG. 23 is a perspective view of a tamper resistant guard having a resilient flap and window covering one jugular infusion site on the neck;

FIG. 24 is a perspective view of a tamper resistant guard having a resilient flap and window covering a second jugular infusion site on the neck;

FIG. 25 is a perspective view of a tamper resistant guard having a resilient flap and window covering a femoral infusion site proximal of a patient's groin;

FIG. 26 is a perspective view of the means for retaining for maintaining the guard in covering relationship over the femoral infusion site;

FIG. 27 is a side view of a tamper resistant guard having a resilient flap and window covering a child's ankle infusion site;

FIG. 28 is a side view of a tamper resistant guard having a resilient flap and window covering an adult's ankle infusion site; and

FIG. 29 is front perspective view of a tamper resistant guard having a resilient flap and window covering an adult's foot infusion site.

### DETAILED DESCRIPTION OF THE INVENTION

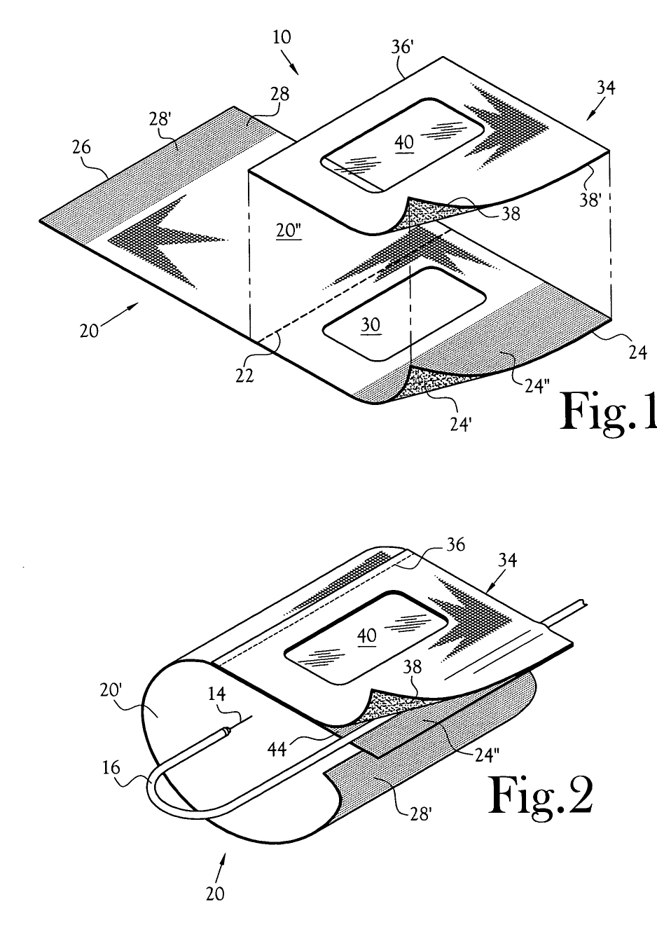

A tamper resistant guard is illustrated at 10 in FIGS. 1 and 2 for covering a portion of the dermal tissue 12 proximal of a patient's forearm infusion site 46 having a flexible catheter 14 inserted therein. The guard device 10 includes a base panel 20 of sufficient size and flexibility to cover an infusion site 46 and dermal tissue 12 proximal of the infusion site. The base panel 20 further includes a receiving end 24, a first retaining end 26, and an open portion 30 therebetween. A resilient flap 34 is hingedly secured at a junction 36 formed by attaching a flap first side 36′ along a medial portion 22 of the base panel 20. The resilient flap 34 includes a flexible window 40 therein that is aligned in register with the panel open portion 30, in order to provide means for rapid visual inspection by a medical practitioner of the covered dermal tissue 12 for indications of infusion site tampering, and/or for indications of infiltration and extravasation of medicinal fluids intended to be transmitted intravenously (vein) or intravascularly (artery or vein), and for indications of symptoms of tissue swelling and degradation and tissue necrosis proximal of the infusion site.

A plurality of embodiments are disclosed herein for configurations and uses of the guard device 10 in order to provide an optimal configuration for a protective cover over each one of a plurality of infusion sites that can be selected by the medical practitioner. Typically at each infusion site, a flexible catheter 14 is inserted through the dermal tissue 12, through the subcutaneous tissue, and into a vein or an artery for intravascular delivery of medications. Depending on the number of infusion sites that are utilized, or have been previously utilized for a patient, a candidate infusion site is selected from a plurality of infusion sites located on the patient's hand, wrist, forearm, elbow, upper arm, neck, upper chest, groin, ankle and/or foot as described herein. A portion of fluid transfer tubing 16 is connected to the inserted catheter 14 for extension under the guard device 10 along a significant distance from the patient to a medication dispensing bag (not shown) that is typically positioned next to the patient or is positioned in an elevated position above the patient.

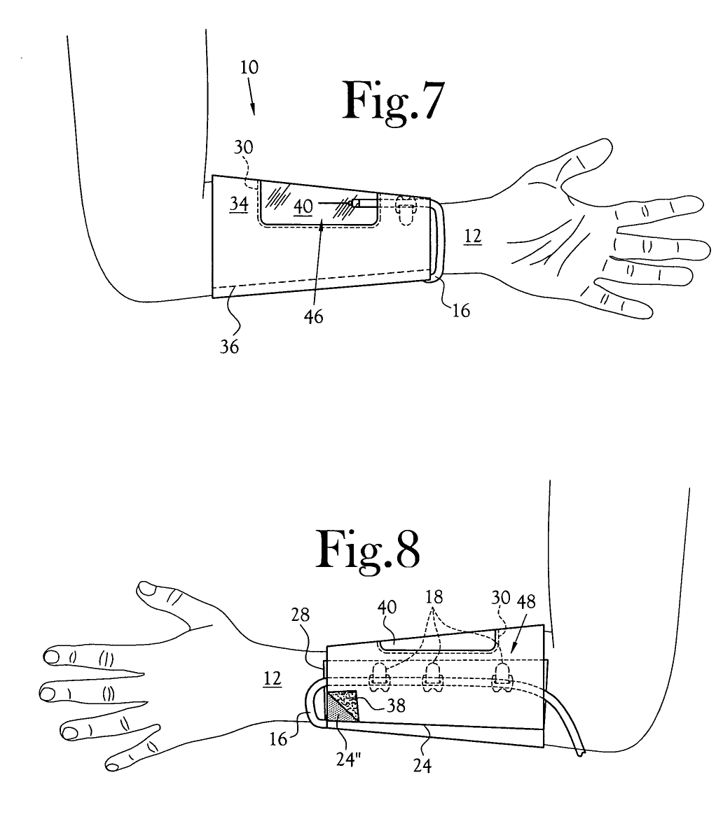

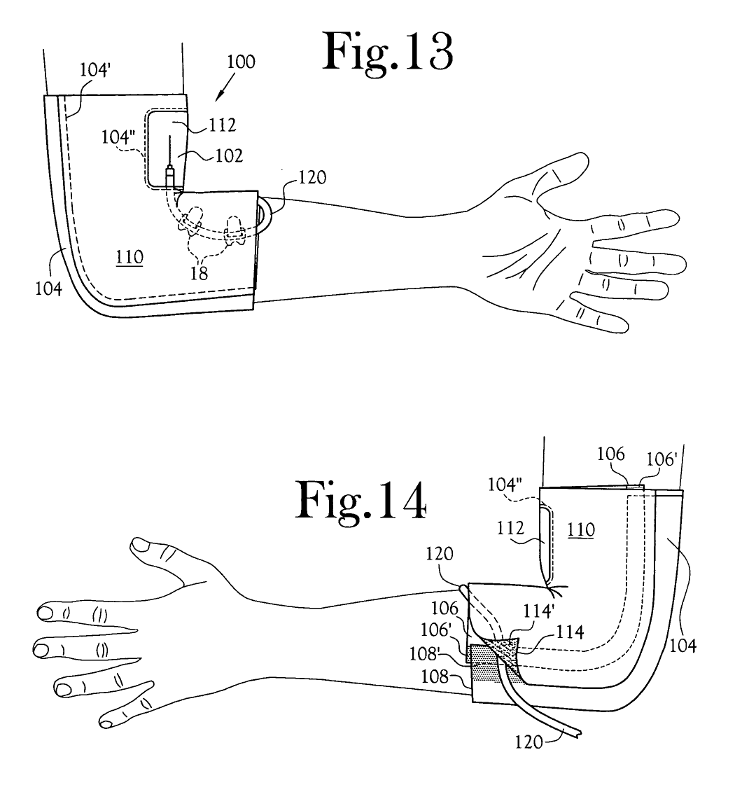

One embodiment of the guard device 10 is illustrated in FIGS. 1-4 and includes an elongated base panel 20 that is positioned to encircle either of the patient's forearms in order to cover a forearm infusion site 46. The base panel 20 is preferably flexible and composed of a stretchable, non-allergenic material having a surface texture that is non-irritating to a patient during extended contact with the patient's skin. The base panel 20 is sufficiently sized to be positioned to encircle the patient's forearm (see FIGS. 7-8), or to cover the patient's elbow and forearm (see FIGS. 9-12), or to cover the patient's upper arm and elbow (see FIGS. 13-16).

One embodiment of the base panel 20 includes a flexible length of material selected from a range of five inches for a child forearm embodiment, to about twenty inches for an adult, with a preferred length of about twelve inches for an adult forearm embodiment. A width for the base panel 20 is selected from a range of about three inches for a child forearm embodiment, to about nine inches for an adult, with a preferred width of about seven inches for an adult forearm embodiment. When the base panel 20 is positioned to encircle the patient's forearm, as illustrated in FIGS. 7 and 8, a dermal surface 20′ contacts the patient's skin. A panel outer receiving surface 20″ includes a medial portion 22 on which a medial seam 36 is formed by hingedly securing a flap first side 36′ on the panel outer receiving surface 20″. Seam 36 is reinforced by stitching and/or by positioning a strip of hook and/or loop fastening material along the width of surface 20″. The base panel 20 includes a receiving end 24, having second means for attaching 24′ disposed on an inwardly oriented, dermal receiving surface 28′. The receiving end 24 further includes an outer faced, first means for attaching 24″ disposed on an outer receiving surface. The receiving end 24 and first means for attaching 24″ are positioned in one embodiment proximal of an outer, distal side of the patient's forearm, with the panel open portion 30 and flap window 40 positioned over the forearm infusion site 46 (see FIGS. 7 and 8). The second means for attaching 24′ includes at least one strip of fastening material having a plurality of loops thereon, in order to releasably fasten with a plurality of hooks 28′ on the panel retaining edge 26. The first means for attaching 24″ includes strips of fastening material having a plurality of hooks for releasably engaging with the flap 34 means for fastening 38. Those skilled in the art will realize that the positioning of the hooks and loops on respective engaging surfaces can be switched.

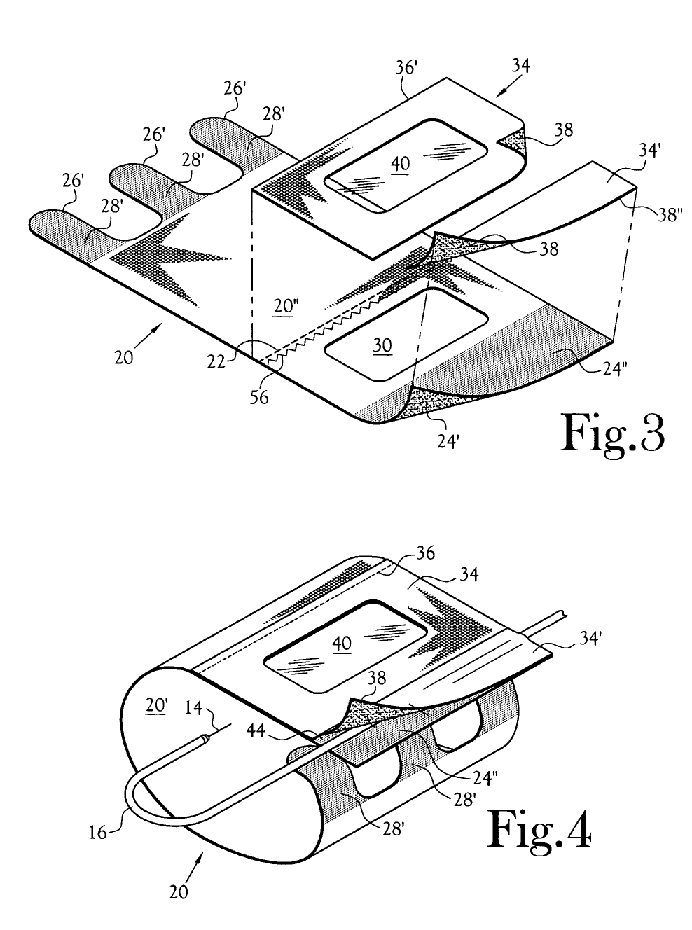

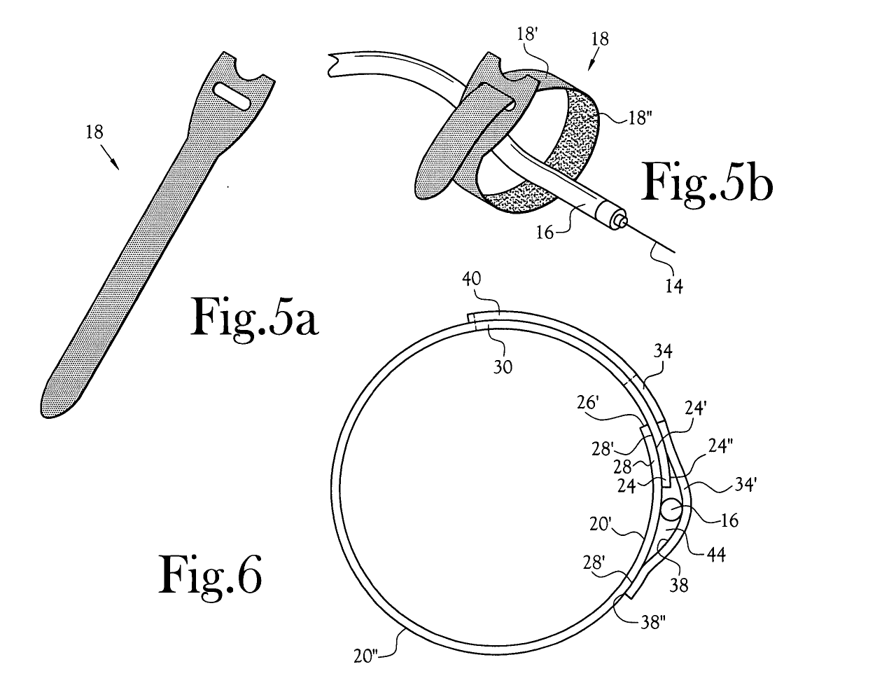

In one embodiment, the base panel 20 serves as a protective cover over the infusion site 46 located on a patient's forearm, with the base panel 20 being extended around the patient's extremity and having a panel retainer edge 26 that is releasably secured by means for retaining against the inner faced surface of the panel receiving end 24. The retainer edge 26 is securely attached under and against receiving end 24 due to means for retaining 28 bonded to the outer faced surface of the panel first end, also referenced as a retainer end 26 (see FIGS. 1 and 2). An alternative configuration including a plurality of panel retainer edges 26′ is illustrated in FIG. 3. The means for retaining 28 is preferably composed of a plurality of hooks 28′ that are releasably attachable to material having a plurality of loops 24′ bonded under the panel receiving end 24. After the medical practitioner has positioned the inserted catheter 14 in the forearm infusion site 46, the base panel 20 is positioned to encircle the forearm and cover the forearm infusion site 46 by releasably fastening the retainer edge 26 to the retainer end 24 at a junction formed against the dermal receiving surface 24′. The junction is positioned preferably on the distal side of the forearm to minimize tampering by the patient (see FIG. 8). Those skilled in the art will recognize that the positioning of the hook and loop fastening materials may be reversed from the disclosed configuration, to provide releasable securement of the dermal receiving surface 24′ with the means for retaining 28 on the retainer edge 26. Regardless of the orientation of the hook and/or loop fastening materials forming the panel means for attaching and panel means for retaining, the panel receiving end 24 and retaining end 26 are releasably attached to each other in an overlapping configuration or an underlying configuration, thereby positioning the base panel 20 in a covering relationship over the forearm infusion site 46.

The guard device 10 further minimizes patient tampering with the forearm infusion site 46 and with tubing 16 extended from the forearm infusion site 46, by the inclusion of the resilient flap 34 that is hingedly attached along the flap first side 36′ proximal of the outer receiving surface 20″ of the base panel 20. The flap 34 provides a unique combination of a plurality of protective features including a centrally oriented flexible and resilient window 40 positioned to cover in register alignment with the open portion 30 of the base panel 20, thereby providing rapid visual inspection of the infusion site while providing at least one layer of protection from intrusion by the patient. The flap 34 includes means for fastening 38 disposed on an inwardly faced surface of a second, distal edge 38′, for covering engagement and releasably securing a selected length of tubing 16 underneath the flap 34.

The flap means for fastening 38 and second edge 38′ are adequately sized to extend past the panel receiving end 24, and to engage in overlapping relationship with both the first means for attaching 24″ and the adjacently positioned hooks and/or loops 28′ of the means for retaining 28 on the panel retainer edge 26. Therefore, the plurality of flap and panel surfaces having hook and loop fastening materials thereon provide a tamper resistant covering orientation 48 forming a gap 44 in which tubing 16 is secured (see FIGS. 6 and 8), between the inwardly faced surface of the flap 34, or a flap extension 34′, and the outer faced first means for attaching 24″ proximal of the receiving end 24 of the base panel 20. An additional protective feature of the guard device 10 includes a second means for attaching 24′ that is releasably secured either directly with hooks and/or loops 28′ of the means for retaining 28, or indirectly by flap extensions 26′ from the means for retaining 28. The guard device 10, when positioned on an extremity, requires a patient to manipulate with one hand the flap means for fastening 38 in one direction to separate the flap from engagement with the panel upper surface first means for attaching 24″, such as in a clockwise direction relative to the extremity or torso. While holding the flap second edge 38′, or flap extension edge 38″, in a disengaged position above the first means for attaching 24″, the patient must manipulate the means for retaining 28 with one hand in an opposing direction relative to the extremity or torso, to separate the means for retaining 28 from the panel second means for attaching 24′ to achieve removal of the flap 34 and base panel 20 from engagement over a typical infusion site 46.

The selected length of tubing 16 is further secured in a generally fixed and distal covering orientation 48 (see FIGS. 6 and 8) between the first flap 34 (see FIG. 2), or the flap extension 34′ (see FIG. 4), by a plurality of easily adjustable ties 18 that are releasably secured within a gap 44 between the underside of the first flap 34 or the flap extension 34′ and the receiving surface 20″ of the base panel 20 (see FIGS. 7-10). The adjustable ties 18 include a hook or a loop fastening material on an outer surface 18′, and on inner surface 18″ having a hook or loop fastening material opposite of the outer surface 18′ to allow each adjustable tie 18 to be separately manipulated to encircle portions of the tubing 16 and to attach to a hook and/or loop surface material on the guard device 10. Each adjustable tie 18 is positioned by the medical practitioner to attach between the outer faced means for attaching 24″ of the base panel 20, and the means for fastening 38 on the first flap 34 or the flap extension 34′. The flap covered adjustable ties 18 thereby provide the medical practitioner with the ability to quickly detach and reattach the tubing 16 within a gap or channel 44 formed between the outer faced means for attaching 24″ proximal of the base panel receiving end 24 and the means for fastening 38 of the flap 34 (see FIGS. 2, 4, and 6).

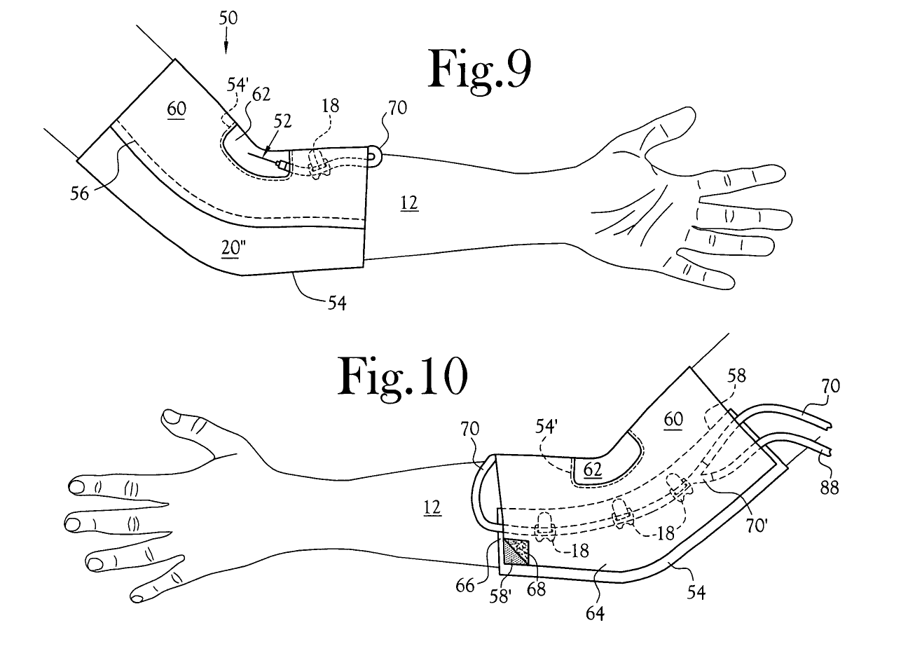

A guard device 50 for covering an antecubital infusion site 52 is illustrated in FIGS. 9 and 10. The guard device 50 includes a base panel 54 that is positioned to encircle either elbow. The base panel 54 includes medial seam 56 that is hingedly attached by stitching, or by utilization of a strip of hook and/or loop fastening material along the width of the panel outer surface 20″, thereby forming a pivotable junction along the seam 56 to allow a medical practitioner to manipulate a resilient flap 60 into a covering relationship on the panel outer receiving surface 20″. A panel first edge 58 is extended in an opposing direction from the junction of seam 56 and the flap 60, in order to encircle the patient's elbow with the base panel 54. A panel first edge 58 includes second means for attaching 58″ on a dermal receiving surface, with the second means for attaching 58″ composed of hook and/or loop material. The panel second means for attaching 58″ is releasably attachable under a panel first edge 58 having first means for attaching 58′ thereon such as hook and/or loop material, in order to securely fasten the panel 54 in an encircling position over the infusion site 52 on the patient's elbow. A panel open portion 54′ is centrally positioned in the base panel 54 and is a sufficiently sized and shaped opening to provide a view of the elbow infusion site 52 when the base panel 54 is in covering engagement. The resilient flap 60 is releasably positioned to cover the panel open portion 54′ and a portion of tubing 70 extended from the infusion site 52. The flap 60 includes a resilient window 62 that is preferably sized and shaped to be aligned in register with the open portion 54′. The open portion 54′ and flexible window 60 are positioned in register to cover the elbow infusion site 52 and a selected portion of the fluid transfer tubing 70 connected to the inserted catheter 14. The flap 60 also includes an attaching side 64 having means for fastening thereon 68, such as hook or loop fasteners, that are releasably secured to the panel first means for attaching 58′ in an encircling configuration over the elbow infusion site 52. The guard device 50 provides a tamper resistant device that covers the elbow infusion site 52 and associated tubing 70, 88 while allowing rapid visual inspection of the infusion site 52 through window 62 and panel open portion 54′ for indications of extravasation of medicine and degeneration leading to atrophy of dermal tissues proximal of the antecubital infusion site 52.

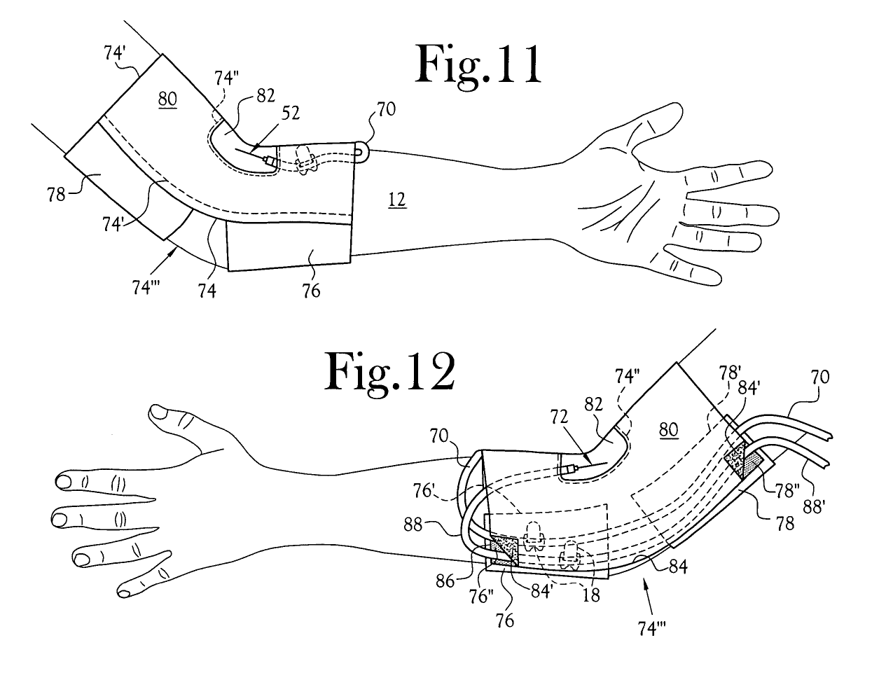

An alternative embodiment of the guard base panel 54 is illustrated in FIGS. 11 and 12 and includes a base panel 74 having a medial seam 74′, an open portion 74″ that is positioned to cover a first antecubital infusion site 52 and a second infusion site 72 proximal of the first antecubital infusion site 52. The base panel 74 further includes a elbow opening 74′″, a first means for retaining 76 and a second means for retaining 78 that are each elongated and flexible. Each means for retaining 76, 78 is extendable from the medial seam 74′ of the base panel 74 to attach at respective distal retaining ends 76′, 78′ to means for attaching 74″ such as hook or loop fasteners attached on a distal side of the base panel 74.

As illustrated in FIG. 11, a resilient flap 80 is hingedly attached by stitching along the medial seam 74′, or by means for fastening such as hook or loop fasteners, to the base panel 74. The flap 80 includes a flexible transparent window 82 therein, and a distal edge 84 having a means for fastening 84′ that is releasably secured to means for attaching 76″, 78″ on the respective panel retaining ends 76′, 78′, thereby maintaining the window 82 in a covering relationship over both infusion sites 52, 72. Flap distal edge 84 provides cover for a portion of tubing 70 and/or a portion of a second tube 88′ extended from the second infusion site 72. The flap distal edge 84 includes means for fastening 84′ thereon, such as hook or loop fasteners, allowing the flap distal edge 84 to be releasably secured over the respective means for attaching 76″, 78″ on the distal retaining ends 76′, 78′. The base panel open portion 74″ and the flexible window 82 are positioned in register to cover the antecubital infusion sites 52, 72. Further, the flap distal edge 84 is releasably secured to cover a selected portion of one or both fluid transfer tubing 70, 88′ connected to the respective infusion sites 52, 72. Therefore, the antecubital guard device 50, including either base panel 54, 74, provides a tamper resistant device that covers an infusion site and associated tubing while allowing rapid visual inspection of the infusion site 52 for indications of extravasation of medications and degeneration and atrophy of dermal tissues proximal of infusion site 52.

A guard device 100 is illustrated in FIGS. 13 and 14 for covering an inner, medial upper arm infusion site 102. The guard device 100 includes a base panel 104 that is positioned to encircle the upper arm and the elbow area of the patient. The base panel 104 is applied around the patient's arm by extending an outer receiving surface having a retaining end 106 and means for retaining 106′ thereon, such as hook or loop fasteners, allowing the receiving end 106 to be releasably secured under a panel receiving end 108 having second means for attaching on a distal side, in order to securely position the base panel 104 to encircle the infusion site 102. A panel open portion 104″ is positioned in the base panel 104 and is sufficiently sized and shaped to provide a view of the upper arm infusion site 102 when the base panel 104 encircles the upper arm. A resilient flap 110 is attached to the base panel 104 along junction 104′, by either stitching or a length of hook and/or loop fastening material. The resilient flap 110 is extended to cover the flap open portion 104″. The flap 110 includes a flexible window 112 sized and shaped to be aligned in register with the panel open portion 104″, thereby positioning the flexible window 112 in covering relationship over the infusion site 102. The flap 110 includes an attaching side 114 having means for fastening 114′ thereon, such as hook or loop fasteners, that are releasably secured to a panel first means for attaching 108′, such as hook or loop fasteners. Flap attaching side 114 is releasably secured in an overlapping relationship with the panel first means for attaching 108′ with a selected length of tubing 120 therebetween, thereby minimizing patient tampering with the selected length of tubing 120 extended from the upper arm infusion site 102.

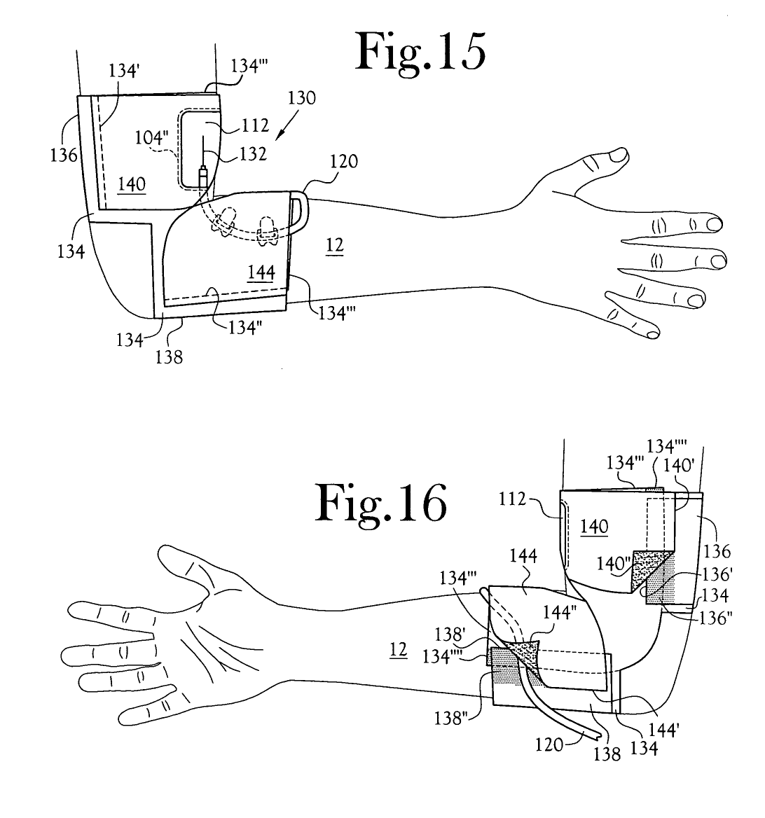

An alternative guard device 130 configured for application on a patient's upper arm and having an elbow opening is illustrated in FIGS. 15 and 16. A base panel 134 is positioned in covering engagement over an upper arm infusion site 132. The base panel 134 includes an upper portion 136 having a distal, receiving end 136′ with upper first means for attaching 136″ thereon, such as hook or loop fasteners. The base panel 134 further includes a lower portion 138 having a distal, receiving end 138′ with lower first means for attaching 138″ thereon. The base panel additionally includes an upper and lower side portions 134′″ having means for retaining 134′″ thereon, for extension to encircle the upper arm and forearm in a direction opposite of the panel upper portion 136, and panel lower portion 138. The encircling portions of the base panel upper and lower side portions 134′″ are releasably joined respectively with upper and lower portions 136, 138, on the medial side of the upper arm as illustrated in FIG. 16. The receiving ends 136′, 138′ have a second means for attaching 138″″ such as hook or loop fasteners (not shown) on a dermal receiving surface of each end 136′, 138′ to releasably attach the respective hook and/or loop fasteners in an overlapping relationship against the medial side of the arm. Two resilient flaps 140, 144 are utilized to encircle the upper arm and the forearm proximal of the patient's elbow. A base end of each flap 140, 144 is hingedly attached at respective junctions 134′ and 134″ by stitching or by means for fastening such as hook or loop fasteners, to respective upper and lower areas of the base panel 134. As illustrated in FIG. 15, the first flap 140 includes at least one flexible transparent window 112 therein, with the window 112 utilized to cover a panel open portion 104″ positioned over the infusion site 132. A first flap attaching side 140′ is releasably secured by a means for fastening 140″ to a panel first means for attaching 136″. A second flap 144 includes a second attaching edge 144′ that is releasably secured by a means for fastening 144″ to a forearm portion having the first means for attaching 138″ thereon. The second attaching edge 144′ is utilized to cover a portion of tubing 120 extended from the infusion site 132. Although not illustrated, the second flap 144 can include a second window therein for protectively covering a second infusion site positioned similar to infusion sites of FIGS. 9-12. Each respective flap distal edge 140′, 144′ includes means for fastening 140″, 144″ thereon, such as hook or loop fasteners, which are releasably secured over the respective means for attaching 136″, 138″ on the panel outer surfaces. The panel open portion 104″ and the flexible window 112 are positioned in register to cover either of the upper arm infusion sites 102, 132. The upper flap 140 and lower flap 144 are releasably secured to cover respective portions of the tubing 120 connected to the either infusion site 102, 132. Therefore, either of the guard devices 100, 130 provide a tamper resistant device that covers an upper arm infusion site and associated tubing. Further, the guard devices 100, 130 allow rapid visual inspection of either upper arm infusion site 102, 132 for indications of extravasation of medications and degeneration and atrophy of dermal tissues proximal of an upper arm infusion site.

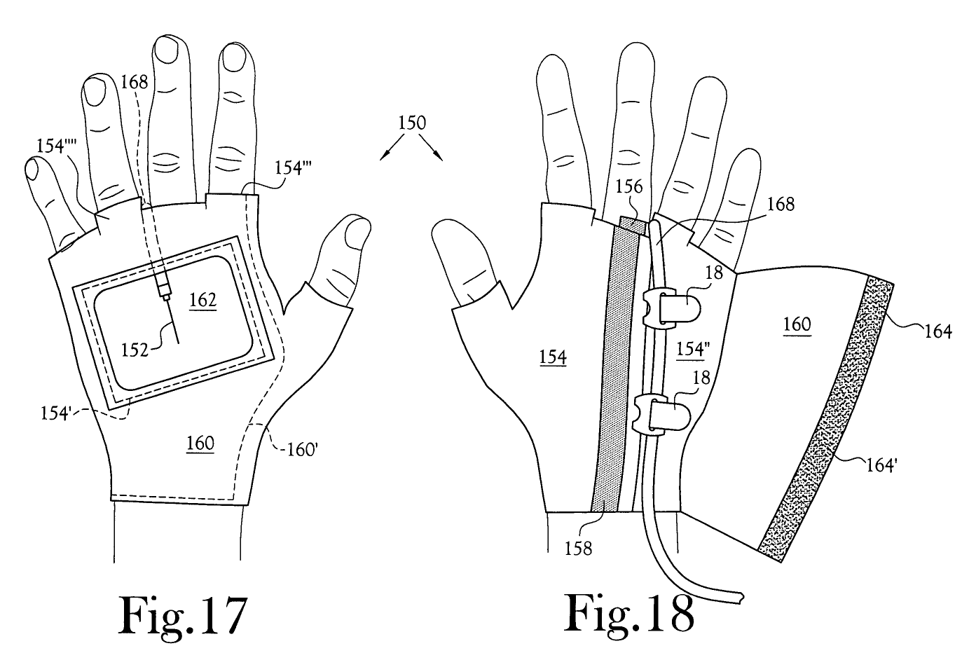

A glove embodiment 150 is illustrated in FIGS. 17 and 18 and includes a glove shaped base panel 154 that is worn to cover a hand infusion site 152. In order to protectively cover the infusion site 152 on the dorsal side of the hand, a resilient flap 160 is utilized in a covering relationship to cover the infusion site 152. The flap 160 is extended from a base junction and seam 160′ that is hingedly attached by stitching proximal of a dorsal portion of the base panel 154. As illustrated in FIG. 17, the dorsal portion of the glove includes at least two finger holes 154′″, 154′″ serving as a binding for maintaining the glove shaped base panel 154 and panel open portion 154′ over the infusion site 152. The base panel 154 includes a first portion having a thumb opening therein, and includes a second portion identified as a base panel palmar portion 154″. Base panel 154 and palmar portion 154″ extend to encircle the patient's hand by releasably connecting proximal of the palm area. The base panel 154 includes a dermal receiving surface having a means for retaining such as hook or loop fasteners thereon, that are releasably engaged with a second means for attaching 156 positioned on a palm edge of the panel palmar portion 154″. The flap 160 includes a flexible transparent window 162 releasably secured in a covering relationship with a dorsal portion of the base panel 154 for maintaining an overlapping relationship of the window 162 in register with the panel open portion 154′ positioned over the infusion site 152. The flap 160 includes an attaching side 164 having means for fastening 164′ thereon, such as hook or loop fasteners, that are releasably secured to the palm first means for attaching 158 to cover the panel palmar portion 154″. A selected length of tubing 168 is releasably secured by a plurality of adjustable ties 18 between the flap 160 and the panel palmar portion 154″, thereby protectively covering the tubing 168 from tampering by the patient.

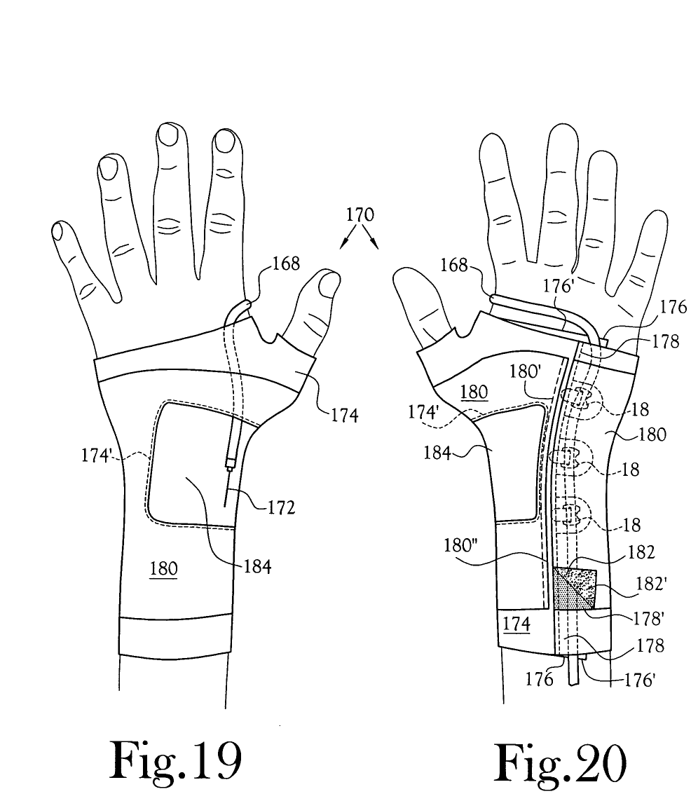

An alternative glove embodiment 170 is provided for covering a carpal and/or wrist vein or artery infusion site 172 is illustrated in FIGS. 19 and 20. A glove shaped base panel 174 includes a thumb opening and an elongated opening through which the patient's fingers extend. The base panel 174 includes an open portion 174′ within the portion of the panel covering the carpal area of the hand. The panel 174 further includes a first outer receiving surface 178 having first means for attaching 178′ thereon. A panel dermal receiving surface 176 includes a second means for attaching 176′ thereon, such as hook or loop fasteners, that are releasably engaged with an underside surface of the first means for attaching 176′ positioned along a palmar area of the patients hand.

A resilient flap 180 is extended in an overlapping relationship with the panel open portion 174′ in order to maintain a flap window 162 covering the carpal infusion site 172. The flap 180 includes an attaching side 182 having means for fastening 182′ thereon, such as hook or loop fasteners, that are releasably secured to the palm first means for attaching 178′ to cover the patient's palm. A selected length of tubing 168 is releasably secured by a plurality of adjustable ties 18 between the flap 180 and the panel receiving end 178, thereby protectively covering the tubing 168 from tampering by the patient. In order to protectively cover the infusion site 172 on the carpal portion of the hand, a resilient flap 180 is positioned in an overlapping relationship over the panel open portion 174′ disposed to cover the infusion site 172. The resilient flap 180 includes a flexible window 184 therein, and includes a flap base end 180′ that is hingedly attached along junction 180″ on the panel outer surface by stitching to secure the flap base end 180′ proximal of the palm portion of the glove 170. As illustrated in FIG. 20, the flap 180 includes an attaching side 182 having means for fastening 182′ thereon, which is releasably secured to first means for attaching 178′ on outer receiving surface 178 of the glove. A selected length of tubing 168 is releasably secured by a plurality of adjustable ties 18 between the flap attaching side 182 and first means for attaching 178′, thereby providing a covering relationship over the tubing 168 to protect from tampering by the patient. Either of the glove guard embodiments 150, 170 provides a tamper resistant device that covers an hand or carpal infusion site and associated tubing while allowing rapid visual inspection of either infusion site 152, 172 for indications of extravasation of medications and degeneration and atrophy of dermal tissues proximal of the infusion sites 152, 172.

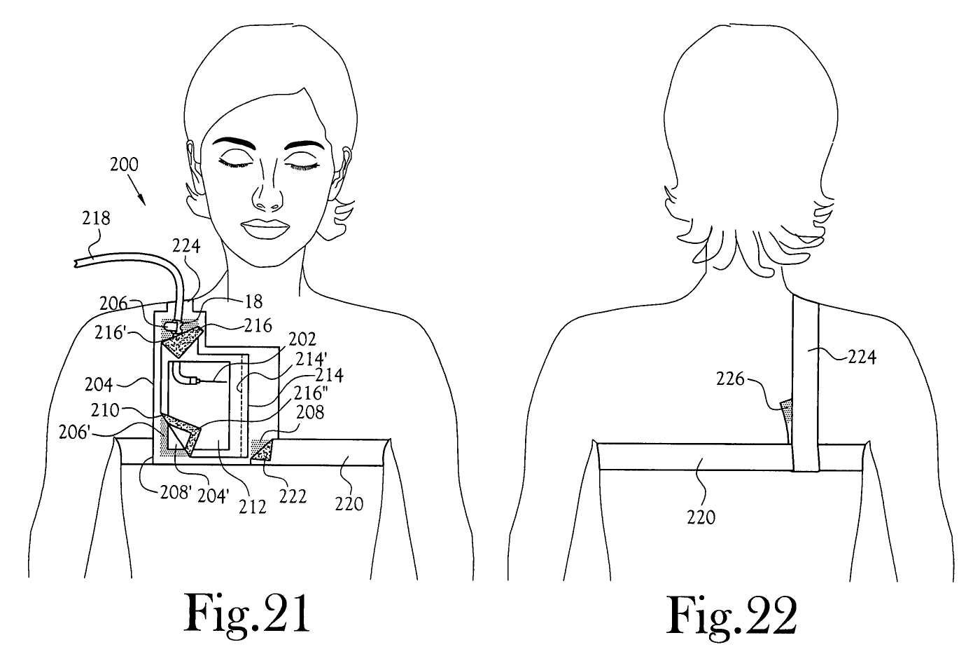

A subclavian embodiment 200 includes positioning a guard device upon the dermal surface of a patient's upper right or upper left portion of his/her chest for covering a subclavian vein or artery infusion site 202 as illustrated in FIGS. 21 and 22. A subclavian base panel 204 is provided having a generally rectangular or pentagonal base of resilient material having an open portion 204′ therein. An upper side 206 of an outer faced panel surface includes means for attaching thereon, such as hook or loop fasteners, and a lower side 208 of the outer faced panel surface includes means for attaching disposed thereon. The base panel 204 includes means for retaining such as at least one torso strap 220 having at least one connecting end 222 with hook or loop fasteners thereon. The torso strap 220 is retained around the patient by extending the strap 220 between the panel lower means for attaching 208 and corner 208′.

As illustrated in FIG. 21, a resilient flap 210 is releasably attached to an outer receiving surface of the panel 204. The resilient flap 210 includes a flexible window 212 therein, with the flap window 212 being positioned in register over the panel open portion 204′ when the panel 204 is positioned over the subclavian infusion site 202. A flap medial portion 214 is hingedly attached by stitching 214′ proximal to the panel lower portions 208, 208′. A flap upper portion 216, also identified as a first attaching side, includes means for fastening 216′ thereon, such as hook or loop fasteners, which is releasably fastened proximal to the panel upper side means for attaching 206. The lower flap perimeter includes means for fastening 216″ thereon, such as hook or loop fasteners, that are releasably secured to the panel lower portion means for attaching 206′. Therefore, the subclavian embodiment 200 provides a tamper resistant guard device that covers an infusion site and associated tubing while allowing rapid visual inspection of the infusion site 202 for indications of extravasation of medications and degeneration and atrophy of dermal tissue proximal of the patient's subclavian infusion site 202.

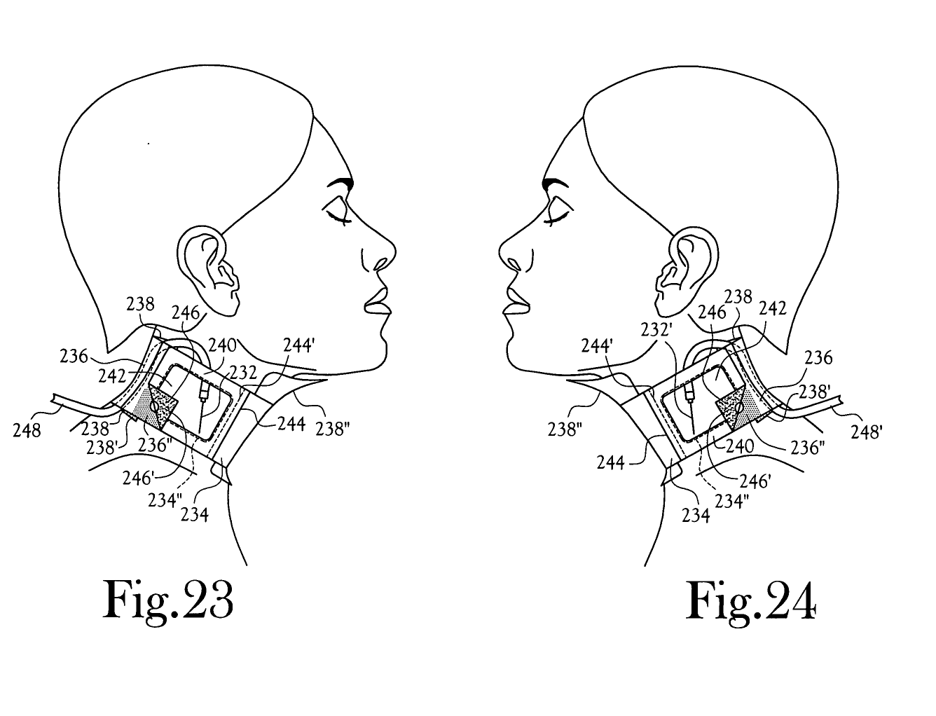

A neck embodiment 230 includes positioning the tamper resistant guard on the patient's neck for covering either of two external jugular vein infusion sites 232, 232′, as illustrated in FIGS. 23 and 24. A base panel 234 includes an elongated length of flexible material having a first end 238 having means for retaining 238′ thereon, such as hook or loop fasteners. A panel receiving end 236 having a first means for attaching 236″ thereon, is extended from a medial portion of the base panel 234 in an opposing direction from the first end 238 in order to encircle the patient's neck. The means for retaining 238′ is attached under the receiving end 236 to a second means for attaching 236′ (not shown), in order to maintain the receiving end 236 proximal of the back of the neck to minimize tampering by the patient. The base panel 234 can be readily repositioned in a mirror-image position on the left neck area of the patient as illustrated in FIG. 24. A panel open portion 234″ is positioned over the right external jugular vein infusion site 232 when the base panel 234 is properly positioned to encircle the patient's neck. A panel means for attaching 236′, such as hook or loop fasteners, is oriented along the base panel 234 proximal to the patient's neck.

Included in the neck embodiment 230 is a resilient flap 240 having means for fastening 246 thereon is releasably secured to the panel means for attaching 236′, in order to securely cover a selected portion of tubing 248, 248′ extended from either of the infusion site 232, 232′. A flap flexible window 242 is positioned in register with the panel opening 234″ when the flap window 242 is securely covering the infusion site 232. The flap 240 is hingedly secured by stitching 244′ at a junction of the flap base edge 244 and the outer receiving surface of the panel 234 by stitching 244′. The flap attaching side 246 having means for fastening 246′ thereon is releasably secured in overlapping relationship on the panel first means for attaching 236. The hinged movement of the resilient flap 240 allows a medical practitioner to lift the flap attaching side 246 without removing the base panel 234 from the patient's neck, thereby allowing a detailed inspection of the dermal tissue proximal of the infusion site and catheter, and allowing disconnecting and reconnecting of tubing 248 extended from the catheter. The means for retaining 238 includes a first panel end of flexible panel material extending from the junction of the panel with flap base edge 244. The means for retaining 238 is positioned to extend around the front of the patient's neck for connection to the panel second means for attaching 236′ (not shown), in order to releasably secure the base panel to the patient's neck. A front neck padding unit 238″ if utilized to maintain the patient's neck and chin in an extended position apart from either of the two jugular infusion sites 232, 232′. Therefore, the neck embodiment 230 provides a tamper resistant device that covers either of the jugular infusion sites 232, 232′ and associated tubing 248, 248′ while allowing rapid visual inspection of either jugular infusion site for indications of extravasation of medications and degeneration and atrophy of dermal tissues proximal of either jugular infusion site 232, 232′.

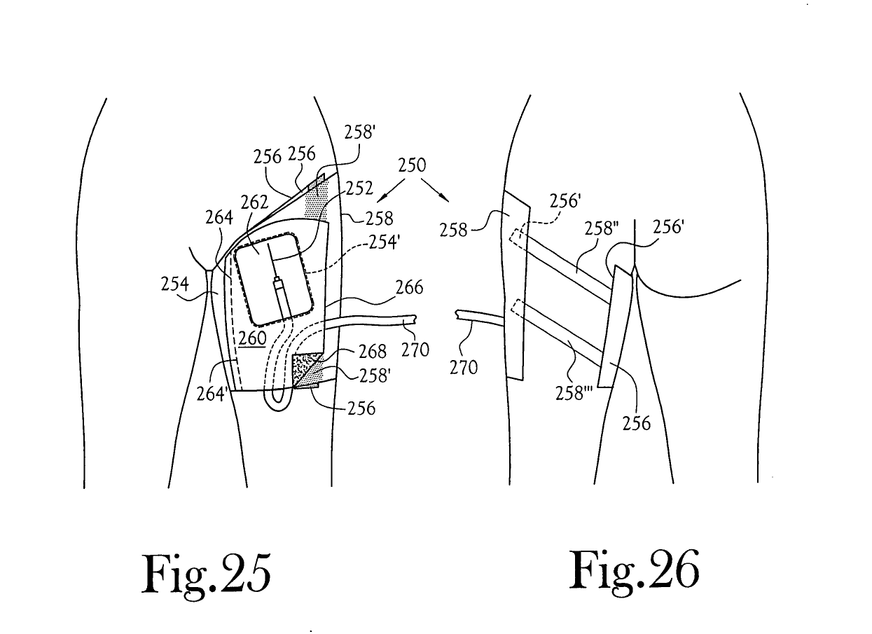

A groin embodiment 250 includes positioning the modified guard device for covering either of a left femoral infusion site 252 as illustrated in FIGS. 25 and 26, or a right femoral infusion site 252′ (not illustrated). A left base panel 254 and a right base panel 254′ are mirror designs, therefore the following discussion is directed to a left side view as illustrated in FIGS. 25 and 26. The base panel 254 includes a panel opening 254′ that is positioned over the femoral artery infusion site 252, and includes a first side panel 256 that partially encircles the patient's thigh. On an inner or outer surface of the first side panel 256, a first means for retaining 256′, such as hook or loop fasteners, is disposed for attachment of each end of a plurality of straps 258′, 258″ that extend around the back of the patient's thigh to attach to a second side panel 258 having additional hook or loop fasteners 256′ on an inner or outer surface (see FIG. 26).

The groin embodiment 250 further includes a resilient flap 260 having an attaching side 266 with means for fastening 268 thereon. The flap attaching side 266 is extended to be releasably secured to the second side panel 258 having first means for attaching 258′ thereon in order for the flap attaching side 266 to cover in overlapping relationship a selected portion of tubing 270 extended from the femoral infusion site 252. A flap flexible window 262 is positioned in register with the panel opening 254″ over the femoral infusion site 252 when the flap attaching side 266 is releasably secured to the first means for attaching 258′. The flap 260 is hingedly secured at a base edge 264 by stitching 264′. The flap means for fastening 268 is oriented along the flap attaching side 266 and is releasable from the panel first means for attaching 258′ to allow a medical practitioner to conduct a detailed inspect of the selected length of tubing 270 and/or the dermal surfaces proximal of the femoral infusion site 252. The groin guard 250 provides a tamper resistant device that covers a femoral infusion site and associated tubing while allowing rapid visual inspection of the femoral infusion site 252 (right femoral site not shown) for indications of extravasation of medications and degeneration and atrophy of dermal tissues proximal of the femoral infusion site 252.

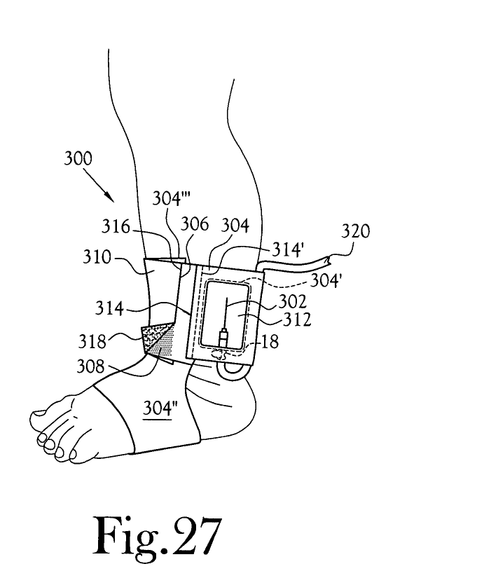

A child's ankle guard embodiment 300 includes positioning a small, child-sized ankle guard for covering a child's ankle infusion site 302 as illustrated in FIG. 27. A base panel 304 includes a panel opening 304′ that is positioned over the child's ankle infusion site 302, and includes a pliable outer receiving end 306 that extends from junction encircles the patient's ankle. A lower portion 304″ of the base panel 304 is utilized to maintain the panel opening 304′ over the child's ankle infusion site 302. The panel receiving end 306 includes a first means for attaching 308, such as hook or loop fasteners positioned on a panel outer receiving surface that are releasably attachable to a means for fastening 318 disposed on an attaching side 316 of a resilient flap 310. A panel first end 304′″ is extendable around the side of the child's ankle opposite the infusion site 302 for connection by a means for retaining (not shown), such as hook or loop fasteners, under the panel receiving end 306.

The flap attaching side 316 with means for fastening 318 thereon is releasably secured to the panel first means for attaching 308 in order for the flap attaching side 316 to securely cover a selected portion of tubing 320 extended from the child's ankle infusion site 302. A flap flexible window 312 is positioned over and in register with the panel opening 304′ when the flap window 312 is securely covering the ankle infusion site 302. The flap 310 is hingedly secured at a base edge 314 by stitching 314′. The flap means for fastening 318 is oriented along the flap attaching side 316 and is releasable from the panel first means for attaching 308 in order to allow a medical practitioner to conduct a detailed inspection of the infusion site 302 and the associated length of tubing 320 sandwiched under the flap attaching side 316. Therefore, the ankle guard 300 provides a tamper resistant device that covers an infusion site and associated tubing while allowing rapid visual inspection of either a right or left ankle infusion site 302 for indications of extravasation of medications and degeneration and atrophy of dermal tissues proximal of the child's ankle infusion site 302.

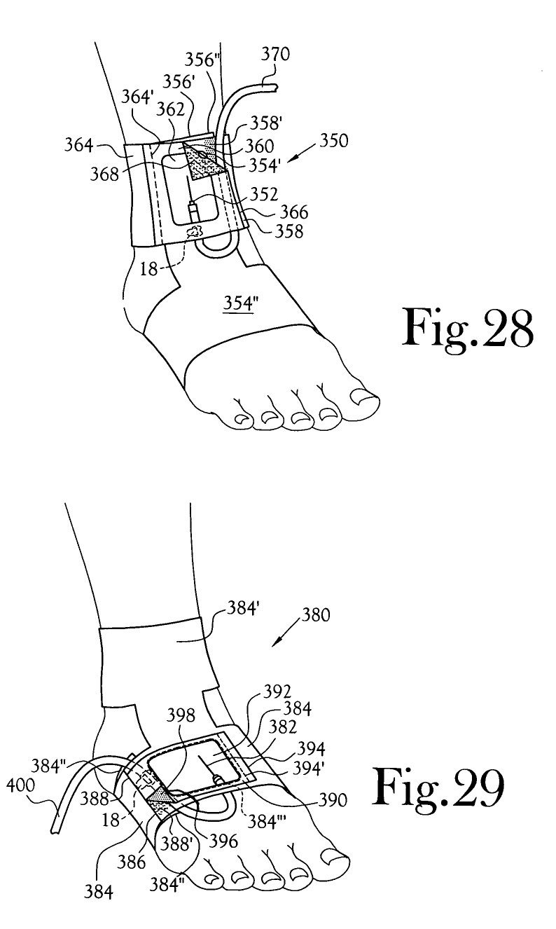

An adult's ankle guard embodiment 350 includes positioning a large sized guard for covering an adult's ankle infusion site 352 as illustrated in FIG. 28. A resilient panel 354 includes a panel opening 354′ that is positioned over the adult's ankle infusion site 352. The panel includes an first end 356′ that is extended against a front portion of the patient's ankle and a lower portion 354″ that is utilized to maintain the panel opening 354′ over the adult's ankle infusion site 352. A panel receiving end 358 extends around the adult's ankle and includes first means for attaching 258′ such as hook or loop fasteners, that are disposed on an outer receiving surface of the panel receiving end 358. The panel receiving end 358 further includes a second means for attaching 358″ (not shown) disposed on the panel dermal receiving surface for releasably attaching to the panel first end 356′ extended around the front portion of the adult's ankle.

A resilient flap 360 having an attaching side 366 with means for fastening 368 thereon is releasably secured to the panel means for attaching 356′, in order for the flap attaching side 366 to securely cover a selected portion of tubing 370 extended from the adult's ankle infusion site 352. A flap flexible window 362 is positioned in register with the panel opening 354′ when the flap window 362 is releasably secured to cover the ankle infusion site 352. The flap 360 is hingedly secured to the base panel 354 at a junction formed by a flap base edge 364 stitched 364′ to the base panel 354. The flap means for fastening 368 is oriented along the flap attaching side 366 and is releasable from the first means for attaching 358 on outer faced panel surface, in order to allow a medical practitioner to conduct a detailed inspection of the infusion site 352 and the selected length of tubing 370 covered by the flap attaching side 366. Therefore, the ankle guard 350 provides a tamper resistant device that covers an ankle infusion site and associated tubing 370 while allowing rapid visual inspection of either left or right ankle infusion site 352 for indications of extravasation of medications and degeneration and atrophy of dermal tissues proximal of the ankle infusion site 352.

An adult's foot embodiment 380 is provided for covering an adult's foot infusion site 382 as illustrated in FIG. 29. A lower base panel 384 is disposed to encircle the patient's foot, and includes an upper panel 384′ that encircles the patients ankle. The lower base panel 384 includes a lower panel first end having means for retaining 384″ thereon, such as hook or loop fasteners, that is positioned to be releasably fastened under a panel receiving end 386. A connecting portion of flexible material extends between the upper panel 384′ and a lower panel 384 having a panel opening 384′″ therein, which is positioned over the adult's foot infusion site 382. The panel receiving end 386 includes a receiving edge 388 having first means for attaching 388′ thereon, such as hook or loop fastening material, that is disposed on the upper side of the lower base panel 384 proximal of the distal side of the patient's foot.

A resilient flap 390 having an attaching side 396 with means for fastening 398 thereon is releasably secured to the panel first means for attaching 388′, in order for the flap attaching side 396 to securely cover in overlapping relationship a selected portion of tubing 400 extended from the adult's foot infusion site 382. A flap flexible window 392 is positioned in register with the panel opening 384′″ when the flap window 392 securely covers the foot infusion site 382. The flap 390 is hingedly secured at a base edge 394 by stitching 394′ to the lower base panel 384. The flap means for fastening 398 is oriented along the flap attaching side 396 and is releasable from the first means for attaching 388′, in order to allow a medical practitioner to conduct a detailed inspection of the infusion site 382 and associated tubing 400 covered by the flap attaching side 396. Therefore, the foot guard 390 provides a tamper resistant device that covers a foot infusion site 382 and associated tubing 400 while allowing rapid visual inspection of the foot infusion site 382 for indications of extravasation of medications and degeneration and atrophy of dermal tissues and tissue necrosis proximal of the infusion site 382.

Those skilled in the art will recognize that the tamper resistant guard provides a resilient base panel that offers a multitude of improved features including a resilient flap having a flexible window therein, with the flap hingedly attached to the base panel in overlapping relationship over the panel open portion that is positioned to cover any one or two adjacent sites of a plurality of infusion sites. The panel and protective cover flap window provides rapid visual inspection of the infusion site for identifying extravasation of medications and degeneration and atrophy of dermal tissues, and tissue necrosis proximal of the infusion site. Further, the tamper resistant guard is readily positioned by the medical practitioner on a plurality of anatomic locations on the patient's body or extremities as illustrated herein. The tamper resistant guard is readily repositioned by the medical practitioner by manipulating and reattaching the appropriate panel means for attaching and means for retaining, thereby providing a secure covering over a new infusion site selected by the medical practitioner when the prior infusion site becomes inadequate. Patient tampering with the infusion site and/or the associated tubing is precluded due to the opposing motions required of the patient to one-handledly remove the flap means for fastening from the panel, and to remove the panel from covering the infusion site by disengaging the outer receiving surface from the panel dermal receiving surface.

Repositioning of the guard device, and adjustment of the intravascular infusion site by a medical practitioner, is rapidly accomplished by disconnecting the panel second means for attaching from the panel means for retaining, and/or manipulation of the window flap attaching side to disconnect from the panel first means for attaching. A medical practitioner can disconnect the infusion tubing, remove the catheter from a first infusion site, and reposition the guard device to cover and protect an adjacent, second infusion site selected by the medical practitioner on the forearm, elbow, upper arm, or on the foot/ankle area. Selection of second or third infusion sites are required when the prior infusion site is in need of rest while the patient receives infusion of medication at another infusion site while tissue degeneration, atrophy and necrosis is treated at the prior infusion site due to extravasation of medicinal fluids. The tamper resistant guard is configured to provide versatility in the positioning of tubing proximal of the infusion site by a plurality of tubing ties protected by covering by the window flap. Those skilled in the art will recognize that the plurality of embodiments illustrated and described herein may not include all uses for the tamper resistant guard, which can be further utilized in alternative configurations on the patient without departing from the spirit and scope of the present invention.

The present invention is illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of applicant's inventive concept.

1. A tamper resistant device for covering an intravascular infusion site and providing visual inspection of the infusion site for indications of infiltration of fluids and disruption of dermal tissues proximal to the infusion site, comprising:
a base panel of sufficient size and flexibility to cover the infusion site and dermal tissue proximal of the infusion site, said base panel is bounded by a first end and a receiving end having first and second means for attaching disposed respectively on an outer receiving surface and a dermal receiving surface, said base panel having an open portion disposed between said panel first end and said receiving end, said open portion is sized to cover without obstructing the infusion site, thereby allowing visual inspection of the infusion site and adjacent dermal tissue without adjustment of said base panel;
means for retaining disposed proximal of said panel first end, said means for retaining is extended to releasably attach to said panel second means for attaching for maintaining said panel positioned with said open portion over the infusion site; and
a resilient flap having a flexible window therein, said window being sized for positioning in register with said panel open portion, said resilient flap having a first side hingedly secured on said panel outer receiving surface, said resilient flap having an attaching side with means for fastening thereon for releasably securing said flap attaching side to said panel first means for attaching with said window positioned in covering relationship over the infusion site.

2. The tamper resistant device of claim 1 wherein said resilient flap attaching side and means for fastening is releasably secured to said panel outer receiving surface in an overlapping relationship with said panel first means for attaching whereby a selected length of tubing extended from the infusion site is retained between said flap attaching side and said panel first means for attaching thereby minimizing patient tampering with said selected length of tubing when covered by said flap attaching side.

3. The tamper resistant device of claim 2 wherein said panel first end having said means for retaining thereon further includes at least one flexible strap releasably engaged between said first end means for retaining and said receiving end second means for attaching, whereby said at least one flexible strap is retained around the patient with said panel open portion maintained over the infusion site and with said flap window positioned in covering relationship over the infusion site.

4. The tamper resistant device of claim 3 wherein said panel first and second means for attaching, said panel means for retaining, and said flap means for fastening are each composed respectively of hook and loop fastening materials, whereby said flap means for fastening on said attaching side is releasably secured to said outer receiving surface of said panel receiving end, and said means for retaining is releasably secured to said dermal receiving surface of said panel, thereby maintaining said flap window in register with said panel open portion positioned over the infusion site.

5. The tamper resistant device of claim 4 wherein said flap fastening means further includes a plurality of ties having hook and loop fastening materials on opposed surfaces of each tie, said ties are releasably attachable between said flap attaching side and said panel outer receiving surface, said plurality of ties are flexibly manipulated to individually encircle said selected length of tubing in a spaced apart orientation, whereby each of said ties encircling said tubing are releasably attached between said flap attaching side and said panel first means for attaching, thereby preventing tampering by the patient with said selected length of tubing maintained between said flap and said panel.

6. A tamper resistant guard for covering an intravascular infusion site while providing visual inspection of the infusion site for indications of infiltration and extravasation of medicinal fluids and degeneration of dermal tissues proximal to the infusion site, comprising:
a base panel having a sufficient size and resiliency to cover the infusion site and dermal tissue proximal of the infusion site, said panel including a first end and a receiving end having first means for attaching disposed on an outer receiving surface and having second means for attaching disposed on a dermal receiving surface of said panel;
an open portion disposed within said base panel, said open portion being sufficiently sized to cover the infusion site when said base panel is positioned over the infusion site, thereby providing visual inspection of the tissue proximal to the infusion site without adjustment of said panel;
means for retaining disposed on a distal end of at least one flexible strap extended from said base panel, said distal end is extendable to encircle a body portion of the patient proximal of the infusion site, said means for retaining is releasably secured to said panel second means for attaching in overlapping relationship thereby maintaining said panel open portion over the infusion site; and
a resilient flap having a flexible window sized for positioning in register with said panel open portion, said resilient flap including an attaching side having means for fastening thereon for releasably securing said flap attaching side to said first means for attaching on said panel outer receiving surface with said window in register with said panel open portion positioned in covering relationship over the infusion site.

7. The tamper resistant guard of claim 6 wherein said resilient flap includes a first side releasably attached in hinged relationship on said base panel outer receiving surface, said attaching side having means for fastening thereon for releasably securing in an overlapping relationship over said first means for attaching on said panel outer receiving surface, said flap attaching side is extended to releasably secure to said panel first means for attaching in an overlapping orientation over a selected length of tubing extended from the infusion site thereby retaining said selected length of tubing between said flap attaching side and said base panel for minimizing patient tampering with said selected length of tubing covered by said flap attaching side.

8. The tamper resistant guard of claim 7 wherein said base panel further including a retaining edge from which said at least two straps means extend, said distal ends having means for retaining thereon are releasably secured to said second means for attaching disposed on said dermal receiving surface, whereby with said base panel is retained on the patient's extremity with said panel open portion and said flap window in covering engagement over the infusion site.

9. The tamper resistant guard of claim 8 wherein said first and second means for attaching of said base panel and said means for fastening of said flap attaching side are composed respectively of hook and loop fastening material whereby said flap attaching side is releasably secured against said base panel outer receiving surface thereby maintaining said flap window in register with said panel open portion positioned over the infusion site.

10. The tamper resistant guard of claim 9 wherein said flap means for fastening further includes a plurality of ties having hook and loop material on opposed surfaces of each tie for releasably attaching each tie against said base panel outer receiving surface, said plurality of ties are flexible and are manipulated to individually encircle said selected length of tubing in a spaced apart orientation, whereby each of said ties encircling said tubing are releasably attached against said first means for attaching disposed on said base panel outer receiving surface with said flap covering said base panel, thereby preventing dislodging of said selected length of tubing from said base panel by patient tampering.

11. A tamper resistant device for covering an intravascular infusion sites on a patient's hand and wrist while providing visual inspection of the infusion site for indications of infiltration and extravasation of medicinal fluids and degeneration of dermal tissues proximal of the infusion site, comprising:
a resilient base panel sized to encircle the patient's hand and wrist in covering engagement over the infusion site, said base panel having first and second means for attaching disposed respectively on an outer receiving surface and a dermal receiving surface, said base panel having an open portion sufficiently sized to cover the infusion site without visually obstructing the infusion site;
means for retaining said base panel in covering engagement over the infusion site, said means for retaining is extendable around the patient's hand and wrist to be releasably secured to said panel second means for attaching on said panel dermal receiving surface; and
a resilient flap having a flexible window sized to be positioned in register with said panel open portion, said resilient flap having an attaching side with means for fastening thereon for releasably securing said flap attaching side to said panel first means for attaching on said outer receiving surface with said window positioned in covering relationship over the infusion site thereby providing visual inspection of the infusion site without adjustment of said window or said base panel.

12. The tamper resistant device of claim 11 wherein said resilient flap includes a first side releasably attached in hinged relationship on said base panel outer receiving surface, said flap attaching side including said means for fastening positioned to be releasably secured in overlapping relationship with said panel first means for attaching disposed on said outer receiving surface to cover a selected length of tubing extended from the infusion site, thereby said selected length of tubing is retained between said flap attaching side and said base panel to minimize patient tampering with said covered selected length of tubing.

13. The tamper resistant device of claim 12 wherein said resilient base panel includes a retaining edge having said means for retaining thereon and further includes a receiving edge having said second means for attaching thereon, said retaining edge is extendable to releasably engage said second means for attaching, whereby with said base panel is retained on the patient's hand and wrist with said panel open portion and said flap window in covering engagement over the infusion site.

14. The tamper resistant device of claim 13 wherein said first and second means for attaching of said base panel and said means for fastening of said flap attaching side are composed respectively of hook and loop fastening material whereby said flap attaching side is releasably secured with said base panel outer receiving surface thereby maintaining said flap window in register with said panel open portion positioned over the infusion site.

15. The tamper resistant device of claim 14 wherein said flap means for fastening further includes a plurality of ties having hook and loop material on opposed surfaces of each tie, said ties are releasably attachable between said flap and said base panel outer receiving surface, said plurality of ties are flexibly manipulated to individually encircle said selected length of tubing in a spaced apart orientation, whereby each of said ties encircling said tubing are releasably attached against said first means for attaching disposed on said base panel outer receiving surface having said flap covering thereon, thereby preventing dislodging of said selected length of tubing from said base panel upon movement by the patient.

16. A tamper resistant guard for covering a portion of a patient's neck having an intravascular site thereon, while providing visual inspection of the infusion site for indications of infiltration and extravasation of medicinal fluids and degeneration of dermal tissues proximal of the infusion site, comprising:
a resilient base panel sufficiently sized to encircle the patient's neck to cover the infusion site and having first and second means for attaching disposed respectively on an outer receiving surface and a dermal receiving surface, said base panel having an open portion sufficiently sized to cover without obstructing the infusion site;
means for retaining said base panel in covering engagement over the infusion site, said means for retaining is extended to be releasably secured with said panel second means for attaching in overlapping relationship with said means for retaining; and
a resilient flap having a flexible window therein, said window being sized for positioning in register with said panel open portion, said resilient flap having an attaching side with means for fastening thereon for releasably securing said flap attaching side to said panel outer receiving surface with said window positioned over the infusion site, thereby providing visual inspection of the infusion site without adjustment of said window or said base panel.

17. The tamper resistant guard of claim 16 wherein said resilient flap includes a first side attached in hinged relationship on said base panel outer receiving surface to provide said flap attaching side having said means for fastening thereon to releasably secured in overlapping relationship to said panel first means for attaching disposed on said base panel outer receiving surface, whereby said flap attaching side is releasably secured to said panel first means for attaching in a covering orientation over a selected length of tubing extended from the infusion site and retained between said flap attaching side and said base panel, thereby said covering orientation of said flap minimizes patient tampering with said selected length of tubing when covered.

18. The tamper resistant guard of claim 17 wherein said resilient base panel includes a retaining edge having said means for retaining thereon and further includes a receiving edge having said second means for attaching thereon, said retaining edge is extendable to releasably engage said second means for attaching, whereby with said base panel is retained on the patient's neck with said panel open portion and said flap window in covering engagement over the infusion site.

19. The tamper resistant guard of claim 18 wherein said first and second means for attaching of said base panel and said flap means for fastening is composed respectively of hook and loop fastening material whereby said flap attaching side is releasably secured with said base panel outer receiving surface thereby maintaining said flap window in register with said panel open portion positioned over the infusion site.

20. The tamper resistant guard of claim 19 wherein said flap means for fastening further includes a plurality of ties having hook and loop material on opposed surfaces of each tie, said ties are releasably attachable between said flap and said base panel outer receiving surface, said plurality of ties are flexibly manipulated to individually encircle said selected length of tubing in a spaced apart orientation, whereby each of said ties encircling said tubing are releasably attached against said first means for attaching disposed on said base panel outer receiving surface having said flap covering thereon, thereby preventing dislodging of said selected length of tubing from said base panel upon movement by the patient.

21. A tamper resistant device for covering a portion of a patient's femoral area having an intravascular infusion site thereon and providing visual inspection of the infusion site for indications of infiltration and extravasation of medicinal fluids and degeneration of tissues proximal of the infusion site, comprising:
a resilient base panel sufficiently sized to cover the infusion site and having first and second means for attaching disposed respectively on an outer receiving surface and a dermal receiving surface, said base panel having an open portion sufficiently sized to cover without obstructing the infusion site, thereby providing visual inspection of the tissue proximal of the infusion site without adjustment of said base panel;
means for retaining extended from said base panel positioned in covering engagement over the infusion site, said means for retaining is releasably secured to said panel second means for attaching positioned in overlapping relationship with said means for retaining, thereby securing said panel open portion over the infusion site; and
a resilient flap having a flexible window therein, said window being sized for positioning in register with said panel open portion, said resilient flap having an attaching side with means for fastening thereon for releasably securing said flap attaching side to said panel outer receiving surface with said window in register with said panel open portion positioned over the infusion site.

22. The tamper resistant device of claim 21 wherein said resilient flap includes a first edge hingedly attached on said base panel outer receiving surface to provide said flap attaching side having said means for fastening thereon to releasably secure in overlapping relationship adjacent said first means for attaching disposed on said base panel outer receiving surface, said flap is releasably secured against said base panel in a covering orientation over a selected length of tubing extended from the infusion site and retained between said flap and said base panel, whereby said covering orientation of said flap minimizes patient tampering with said selected length of tubing when covered by said flap.

23. The tamper resistant device of claim 22 wherein said resilient base panel includes a retaining edge having said means for retaining thereon and further includes a receiving edge having said second means for attaching thereon, said retaining edge is extendable to releasably engage said second means for attaching, whereby with said base panel is retained on the patient's hand and wrist with said panel open portion and said flap window in covering engagement over the infusion site.

24. The tamper resistant device of claim 23 wherein said first and second means for attaching of said base panel and said flap means for fastening are composed respectively of hook and loop fastening material whereby said flap attaching side is releasably secured with said base panel outer receiving surface thereby maintaining said flap window in register with said panel open portion positioned over the infusion site.

25. The tamper resistant device of claim 24 wherein said flap means for fastening further includes a plurality of ties having hook and loop material on opposed surfaces of each tie, said ties are releasably attachable between said flap and said base panel outer receiving surface, said plurality of ties are flexibly manipulated to individually encircle said selected length of tubing in a spaced apart orientation, whereby each of said ties encircling said tubing are releasably attached against said first means for attaching disposed on said base panel outer receiving surface having said flap covering thereon, thereby preventing dislodging of said selected length of tubing from said base panel upon movement by the patient.

26. A tamper resistant guard for covering a portion of a patient's foot and ankle having an intravascular infusion site thereon and providing visual inspection of the infusion site for indications of infiltration and extravasation of medicinal fluids and degeneration of dermal tissues proximal of the infusion site, comprising:
a resilient base panel sufficiently sized to cover the infusion site and having first and second means for attaching disposed respectively on an outer receiving surface and a dermal receiving surface, said base panel having an open portion sufficiently sized to cover without obstructing the infusion site, thereby providing visual inspection of the tissue proximal of the infusion site without adjustment of said base panel;
means for retaining extended from said base panel positioned in covering engagement over the infusion site, said means for retaining is releasably secured to said panel second means for attaching positioned in overlapping relationship with said means for retaining, thereby securing said panel open portion over the infusion site; and
a resilient flap having a flexible window therein, said window being sized for positioning in register with said panel open portion, said resilient flap having an attaching side with means for fastening thereon for releasably securing said flap attaching side to said panel outer receiving surface with said window positioned over the infusion site.

27. The tamper resistant guard of claim 26 wherein said resilient flap includes a first edge hingedly attached on said base panel outer receiving surface to provide said flap attaching side having said means for fastening thereon to releasably secure in overlapping relationship adjacent said first means for attaching disposed on said base panel outer receiving surface, said flap is releasably secured against said base panel in a covering orientation over a selected length of tubing extended from the infusion site and retained between said flap and said base panel, whereby said covering orientation of said flap minimizes patient tampering with said selected length of tubing when covered by said flap.

28. The tamper resistant guard of claim 27 wherein said resilient base panel includes a retaining edge having said means for retaining thereon and further includes a receiving edge having said second means for attaching thereon, said retaining edge is extendable to releasably engage said second means for attaching, whereby with said base panel is retained on the patient's foot and ankle with said panel open portion and said flap window in covering engagement over the infusion site.

29. The tamper resistant guard of claim 28 wherein said first and second means for attaching of said base panel and said flap means for fastening are composed respectively of hook and loop fastening material whereby said flap attaching side is releasably secured with said base panel outer receiving surface thereby maintaining said flap window in register with said panel open portion positioned over the infusion site.

30. The tamper resistant guard of claim 29 wherein said flap means for fastening further includes a plurality of ties having hook and loop material on opposed surfaces of each tie, said ties are releasably attachable between said flap and said base panel outer receiving surface, said plurality of ties are flexibly manipulated to individually encircle said selected length of tubing in a spaced apart orientation, whereby each of said ties encircling said tubing are releasably attached against said first means for attaching disposed on said base panel outer receiving surface having said flap covering thereon, thereby preventing dislodging of said selected length of tubing from said base panel upon movement by the patient.
